(19) United States
(12) Patent Application Publication
(10) Pub. No.: US 2012/0283419 A1
(43) Pub. Date: Nov. 8, 2012

(54) Separation of protein monomers from aggregates by solid weak anion exchange support functionalized with amine moieties

(75) Inventors: Bhaktavachalam Thiyagarajan, Bethleham, PA (US); Wei Guo, Bethlehem, PA (US); Nandu Deorkar, Cedar Knolls, NJ (US)

(73) Assignee: Avantor Performance Materials, Inc.

(21) Appl. No.: 13/373,064

(22) Filed: Nov. 3, 2011

Related U.S. Application Data
(60) Provisional application No. 61/518,305, filed on May 3, 2011.

(51) Int. Cl. C07K 1/18; C07K 16/00

(52) U.S. Cl. 530/389.1; 530/416

(57) ABSTRACT

A flow-through process for separating protein monomer from aggregates of that protein in a solution containing both protein monomer and aggregates of that protein, the process includes the steps of

1) contacting the solution at a pH of from 4 to 7 with a weak anion exchange media comprised of multiple primary, secondary and/or tertiary amine functionalization groups whereby the protein monomer flows through the media without binding thereto and the aggregates are retained on the media, and

2) collecting the flow-through as purified protein monomer.

### FIELD OF THE INVENTION

The invention relates to using solid weak anion exchange support functionalized with substituted polyamine that provide multiple primary, secondary and/or tertiary amine groups for the separation and/or purification of proteins, such as IgG (antibody) monomers, from their aggregates. This invention differs with the currently available chromatographic media in that the chromatographic media used in this invention is effective in removal of antibody aggregates to give pure protein monomers under flow-through conditions in a single step at different pH's whereas currently available anion exchange and cation exchange media separate protein monomers from their aggregates based on much less effective bind-elution methodology.

### BACKGROUND OF THE INVENTION

In today's medical portfolio of products proteins, especially immunoglobulins, play an important part. However, many proteins may undergo numerous physical and chemical changes during manufacturing, shipping, and storage that can adversely alter drug potency and safety. Earlier concerns focused upon de-naturation, and oxidation of protein drugs. However, aggregation has emerged as a key issue for peptide- or protein-based therapeutics, including loss of efficacy, altered pharmacokinetics, reduced stability and product shelf life, and induction of unwanted immunogenicity. As a result, regulatory agencies have increased their scrutiny of protein purification, especially a regards to aggregation, and in response biopharmaceutical companies have increased their efforts to control the amount of aggregates in the final product. In particular, removal of aggregated protein, such as aggregated immunoglobulins, for example aggregated IgG, is the most critical requirement before the protein can be used for therapeutic purpose.

The removal of aggregated proteins from their monomers has primarily heretofore been carried out by size exclusion chromatography (SEC) because SEC separates proteins based on their size and size of the aggregates are much larger than the protein monomers. Although SEC is now the most commonly employed method for aggregates detection and separation in analytical scale, the application of SEC is often limited, especially in large scale process, because of its low efficiency and small sample loading capacity and therefore increases the processing time and hence cost of the production. With the ever quickening, fast development of bioengineering processes, therapeutic proteins, especially IgG, are now produced in the scale of several grams per liter, and an efficient method of aggregates removal is needed now more then ever.

Cation exchange chromatographic media from several manufactures such as Fractogel® EMD (EMD Chemicals), POROS® (Applied Biosystems), Capto S® (GE Healthcare Life Sciences), Toyopearl® Sp-650 C (Tosoh Bioscience), PolyABx (Avantor Performance Materials, Inc. formally Mallinckrodt Baker, Inc.) and PolyCSx (Avantor Performance Materials, Inc.) have been successfully used to separate IgG monomers from aggregates at lower pH (about 4-7). Similarly, anion exchange chromatographic media such as Fractogel® EMD DEAE-650 (EMD Chemicals), Source Q and Q Sepharose® (GE Healthcare Life Sciences) were also used for removal of aggregates from IgG monomers at higher pH (about 6-9) according to WO 99/62936. However, they work on a bind and elute method wherein bound monomers are eluted using buffer containing ˜0.5 M sodium chloride or other salts. Various problems and limitations are encountered with the bind and elute methodology that limits its usefulness. Volume throughput is significantly limited such that the capacity for the methodology is a severe limitation. The methodology requires buffer to release the bound monomer and the use of buffer for this purpose is not desired. There is therefore a need for a flow-through methodology for separation of protein monomers from protein aggregates wherein the desired protein monomer flows through the separation media and the protein aggregate and impurities is bound to the solid media.

### SUMMARY OF THE INVENTION

In the current invention, from a solution containing both protein monomer and aggregates of that protein, protein aggregates are removed from protein monomer groups under flow-through process conditions at a pH of from pH 4 to 7 by employing weak anion exchanger solid media (an immobile matrix) comprised of multiple primary, secondary and/or tertiary amine functionalization groups obtained by functionalizing a solid support with a substituted polyamine, such as for example polyethyleneimine, polyallylamine, and/or polyalkyleneamine.

The present invention discloses a novel procedure for the removal of protein aggregates, such as IgG aggregates, from protein monomers, such as IgG monomer, under flow-through conditions at pH 4 to 7 by using a weak anion exchange solid support functionalized with substituted polyamine that provide multiple primary, secondary and/or tertiary amine groups. The functionalization of the weak anion exchange solid support is accomplished with a suitable polyamine, such as for example, polyethyleneimine, polyallylamine and polyalkylene polyamines. For example, a solution of a mixture of IgG monomer and IgG aggregates is passed through a column packed with weak anion exchanger, namely PolyPEI, and the monomer IgG passes through the column without any interaction, while the IgG aggregates bind to the column and can the bound aggregates be removed using high salt buffer and the column is then ready for second injection. Thus, in accordance with this invention a solution sample containing a mixture of protein and protein aggregates is passed through a solid weak anion exchanger media functionalized with multiple primary, secondary and/or tertiary amine groups and the protein monomer flows through the solid media without binding thereto while the protein aggregate binds to the solid media resulting in a purified protein monomer product being collected from the portion of the sample flowing through the solid media.

A further aspect of the invention resides in the use of a purification buffer wherein the pH of the purification buffer is chosen so that its pH is less than the pI (Isoelectric point) of the protein being separated, and preferably the pH of the purification buffer is below the pI of the protein being separated. Under this condition, flow through fractions obtained using this chromatographic media contains only monomers. The purification can be performed using purification buffer having conductivity of less than 10 mS/cm and preferably less than 5 mS/cm. Separation under these conditions gives protein monomer having a purity of more than 95% and a yield of more than 85% at varying pH and loading concentrations.

Further embodiments of the invention include, but are not limited to the following embodiments. The process is conducted at a pH of from pH 5.5 to 6.5. The process is conducted at a pH that is less than the pI of the protein monomer being separated. The process is conducted at a pH that is from 1 to 2 units below the pI of the protein monomer being separated. The solution contacting the media contains a purification buffer having a conductivity of less than 10 mS/cm. The weak anion exchange media comprised of multiple primary, secondary and/or tertiary amine functionalization groups is obtained by functionalizing a solid support with a substituted or unsubstituted polyamine, such as a polyamine selected from polyethyleneimine, polyallylamine, and polyalkyleneamine. The weak anion exchange media comprised of multiple primary, secondary and/or tertiary amine functionalization groups preferably silica or polymethacrylate solid media functionalized with the substituted or unsubstituted polyamine. The protein monomer being separated from aggregates thereof is an immunoglobulin, preferably IgG. The weak anion exchange media functionalized with multiple primary, secondary and/or tertiary amine groups is located in a column and the sample solution of protein monomer and aggregates thereof is injected into the column. An additional step of removal of the bound aggregates and other impurities with a high conductivity buffer preferably a buffer having a conductivity of>60 mS/cm at the same pH is conducted following collecting of the purified protein monomer.

### BRIEF DESCRIPTION OF THE DRAWINGS

In the drawings:

FIG. 1 is a chart of the SEC analysis of IgG monomer and its aggregates generated according to the methodology described in the Preparation Example;

FIG. 2 is a chart of the SEC analysis of IgG monomer and its aggregates separated according to Example 1;

FIG. 3 is a chart of the SEC analysis of certain collected fractions collected in Example 1 and charted in the graph of FIG. 2;

FIG. 4 is a chart of the SEC analysis of the diluted injection sample of Example 2;

FIG. 5 is a chart of the SEC analysis of IgG monomer and its aggregates separated according to Example 2;

FIG. 6 is a chart of the SEC analysis of certain collected fractions collected in Example 2 and charted in the graph of FIG. 5;

FIG. 7 is a chart of the SEC analysis of IgG monomer and its aggregate separated according to Example 3;

FIG. 8 is a chart of the SEC analysis of the IgG monomer and its aggregates separated according to Example 4;

FIG. 9 is a chart of the SEC analysis of certain collected fractions collected in Example 3 and charted in the graph of FIG. 8;

FIG. 10 is a chart of the SEC analysis of the IgG monomer and its aggregates separated according to Example 5;

FIG. 11 is a chart of the SEC analysis of the IgG monomer and its aggregates separated according to Example 6;

FIG. 12 is a chart of the SEC analysis of certain collected fractions collected in Example 3 and charted in the graph of FIG. 11;

FIG. 13 is a chart of the SEC analysis of the IgG monomer and its aggregates separated according to Example 7;

FIG. 14 is a chart of the SEC analysis of the IgG monomer and its aggregates separated according to Example 8;

### DETAILED DESCRIPTION OF THE INVENTION

The subject of this invention concerns purification of IgG monomers from its aggregates under flow-through conditions mode at a pH of from pH 4 to 7 using solid weak anion exchange support functionalized with substituted polyamine that provide multiple primary, secondary and/or tertiary amine groups. The functionalization of the solid media to provide the primary, secondary and/or tertiary amine groups thereon can be by use of any suitable polyamine, such as for example, a polyamine such as polyethyleneimine and polyallylamine. The amine functionalized media can be based on any suitable inorganic media such as for example silica gel, titanium dioxide, and zirconia, or any suitable organic media such as polymethacrylate and agarose. In addition, such solid functionalized support could be of particles with particle diameters of from about 3 microns to about 500 microns and can be used in a packed column or could be embedded into a membrane. Such solid weak anion exchange support media functionalized with polyamines to provide multiple primary, tertiary or secondary amines groups are commercially available from Avantor Performance Materials, Inc. For example, a suitable polyethyleneimine based functionalized polymethacrylate media is sold under the product name BakerBond® PolyPEI with the product code PN 7585 by Avantor Performance Materials, Inc. Others of such media are disclosed in US Patent Application Publication No. 2008/0203029 A1 the disclosure of which is incorporated herein. Additionally, several polyethyleneimine functionalized silica media product is also available from Avantor Performance Materials, Inc. with different particle size and pore size, particularly those having a particle size of from 3 to 70 microns and with a pore size of from 50 to 1000 Å, such as those disclosed in U.S. Pat. No. 4,551,245, the disclosure of which is incorporated herein.

The separations are carried out been pH 4 and 7, preferably between 5.5 and 6.5, so that protein monomers are not negatively charged and therefore would not bind to the anion exchange media. The protein monomers pass through the media during sample injection process, while the protein aggregates bind to the column until further elution or cleaning using a high salt buffer.

IgG aggregates used in the examples were generated by heating a concentrated human IgG monomer solution for about 15 to 20 min at about 65° C. The produced mixture contains both IgG monomer and aggregates in about 1:1 ratio as analyzed by Size Exclusion Chromatography (SEC) and the sample was filtered through a 0.45 um membrane before injection. In order to match the conditions close to that of real condition expected in antibody purification, fresh monomer IgG was added to change the concentration of aggregates. The methods introduced with this invention are applicable over a wide range of pH and conductivity.

The examples were carried out using a flow-though mode. The IgG monomer and aggregates mixture was pumped through a pre-equilibrated column packed with PolyPEI media. The monomer passes through the column without retention while the aggregates bind to the column. After all the sample solution passes through the column, the column was regenerated by washing with a high salt buffer solution of conductivity 60 mS/cm or more.

In one embodiment, a purification of IgG monomer from a mixture of IgG monomer containing about 8% IgG aggregates was accomplished by passing the sample through a column packed with the weak anion exchanger, PolyPEI at pH 6.0 using a purification buffer having a conductivity of less than 10 mS/cm and collecting purified IgG monomer in the flow-through followed by removal of the bound IgG aggregates and other impurities with a high conductivity buffer (>60 mS/cm) at the same pH. In another embodiment, monomer IgG was purified from a mixture of IgG monomer and aggregates containing about 50% IgG aggregates using the same procedure. Under ideal chosen conditions, the flow-through fraction contains only IgG monomer and no detectable amount of aggregates as determined by Size exclusion chromatography (SEC).

By protein as used in the application it means any macromolecule comprising one or more polypeptide chains capable of forming aggregates, such as for example, immunoglobulins and bovine serum albumin. By monomeric form of a protein it is meant that the protein molecule(s) is not associated with a second or more protein molecule either covalently or non-covalently. By protein aggregates it is meant protein molecule is associated with a second or more protein molecule(s) either covalently or non-covalently. By flow-through mode it is meant that the purification method is conducted in a manner such that the sample containing both protein monomer and aggregates is brought into contact with the exchange media and the desired monomeric form of the protein does not bind to the media but flows through the media and is collected a flow-through purified protein monomer product while aggregates of the protein are retained on the media.

The invention is illustrated by, but not limited to, the following examples.

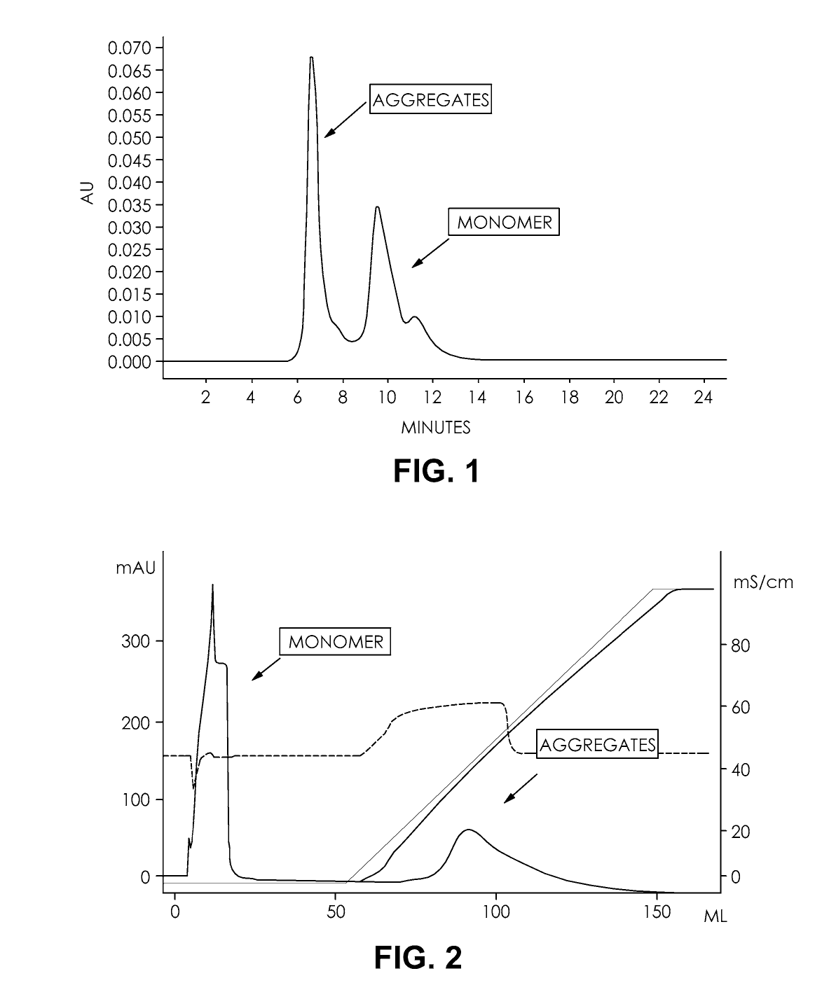

Preparation and analysis of IgG aggregates and characterization thereof. To make IgG aggregates, 150 mg of human IgG (Sigma PN G4386) was dissolved in 10 ml solution containing 100 mM NaCl and 100 mM sodium phosphate at pH 7.2. The protein solution was then heated at 64° C. for 20 min. The protein solution was cooled down and refrigerated for further use. A size exclusion chromatography (SEC) column (Wyatt narrow pore 300×4 mm ID) was equilibrated with a solution containing 400 mM sodium perchlorate and 100 mM sodium phosphate at pH 6.7. 20 microliters sample solution containing IgG monomer or IgG aggregates or both could be injected, and the analysis was carried out at a flow rate of 0.35 ml/min and the typical SEC is shown in FIG. 1.

### Example 1

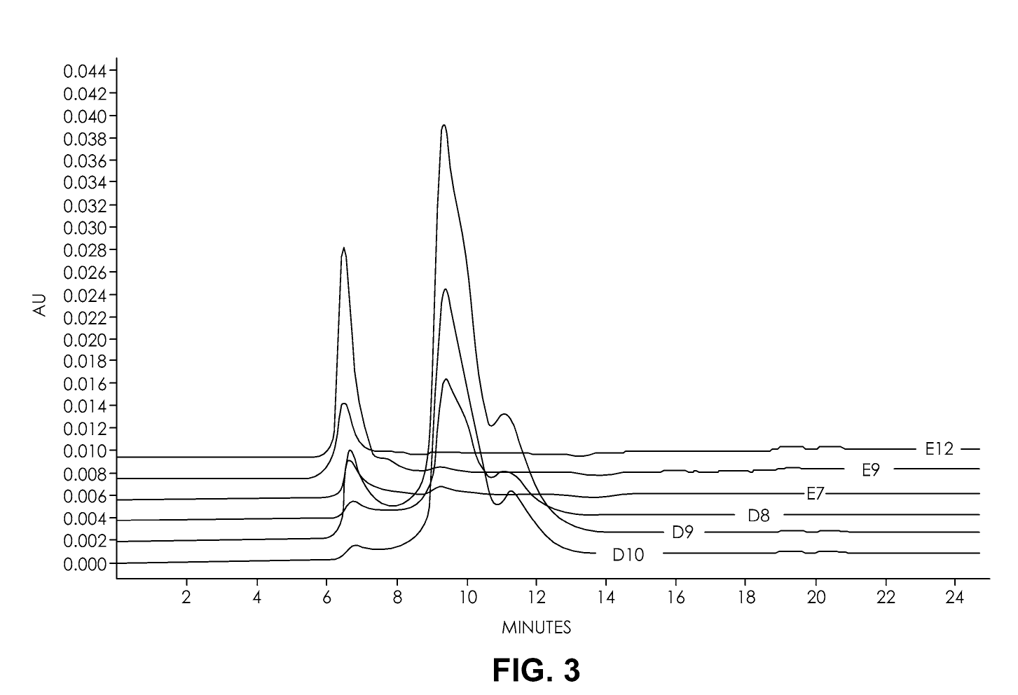

A column (100×7.75 mm ID) was packed with 35 micron BakerBond® PolyPEI. The column was equilibrated with 50 mM MES (2-(N-morpholino)ethanesulfonic acid) pH 6.0 buffer (Equilibration buffer) for at least 8 column volumes (CV). 12 ml of the solution containing 0.75 mg/ml IgG and 0.75 mg/ml IgG aggregates in Equilibration buffer was filtered through a 0.45 μm membrane filter and then injected into the pre-equilibrated PolyPEI column at a flow rate of 2.0 ml/min. After the injection, the column was washed with Equilibration buffer for 5 CV and the flow-through product collected. Then the retained IgG aggregate was washed out of the column with Equilibration buffer containing 1.0 M NaCl. All fractions were collected for further analysis by SEC chromatography as described in the section titled “Preparation and analysis of IgG aggregates and characterization thereof”. The SEC analysis of the separated monomer and aggregate solutions and the fractions thereof are shown in FIGS. 2 and 3.

### Example 2

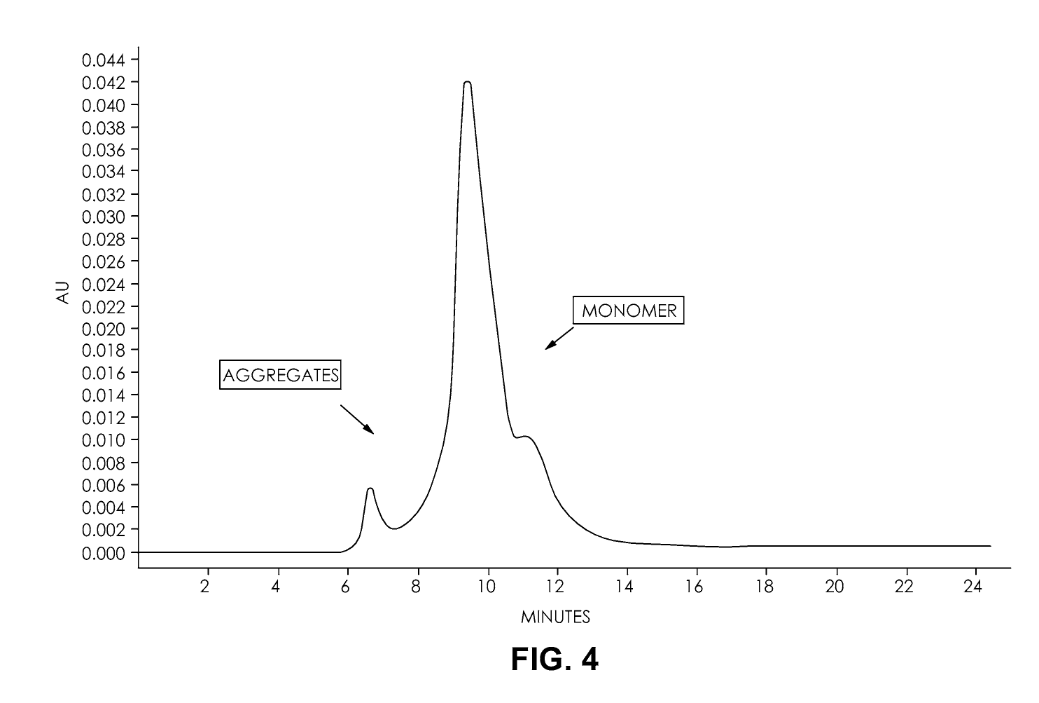

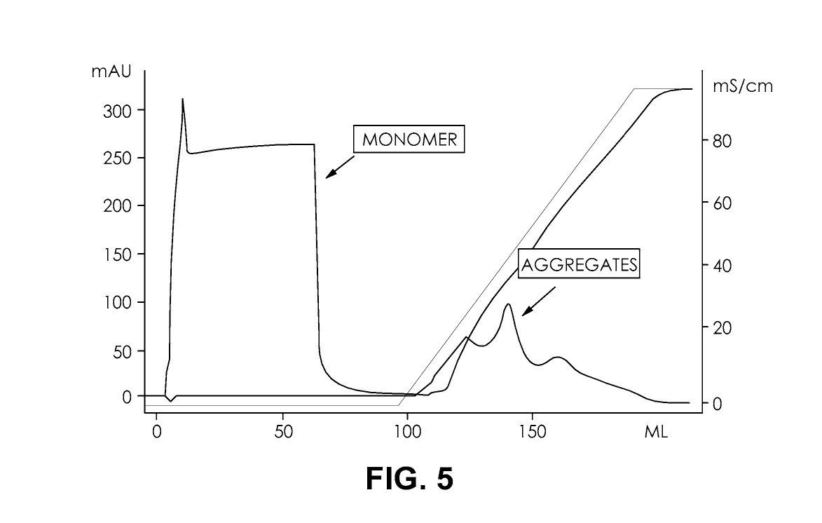

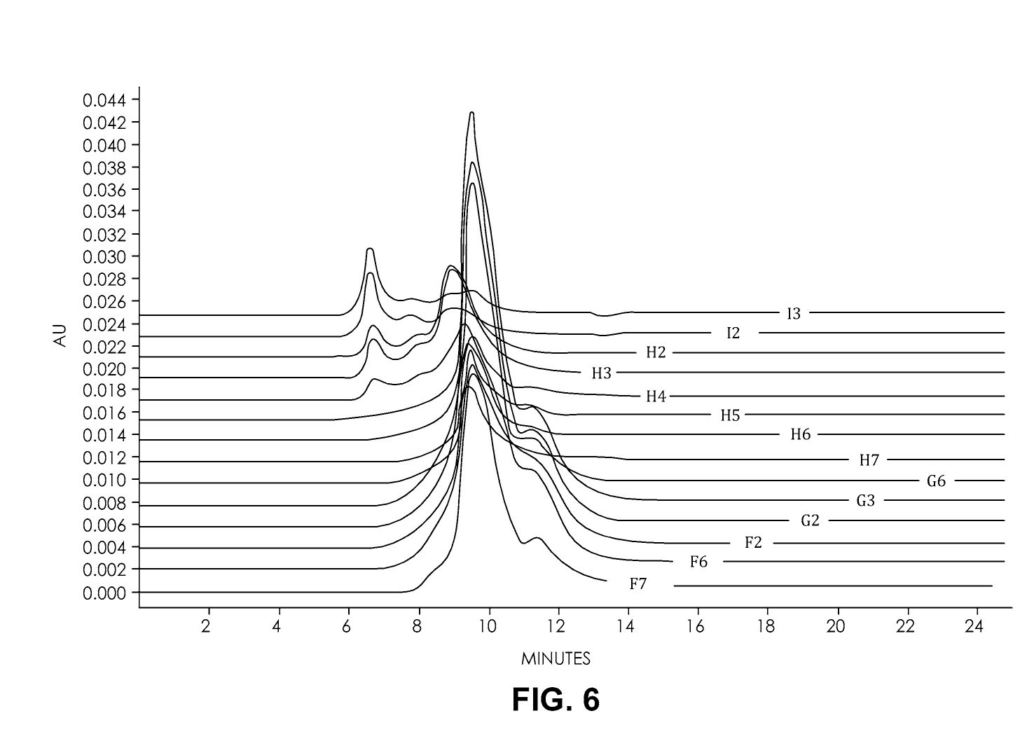

A column (100×7.75 mm ID) was packed with 35 micron BakerBond® PolyPEI. The column was equilibrated with 50 mM MES pH 6.0 buffer (Equilibration buffer) for at least 8 column volumes (CV). 60 ml of the solution containing 1.08 mg/ml IgG monomer and 0.08 mg/ml IgG aggregates in Equilibration buffer was filtered through a 0.45 μm membrane filter and then injected into the pre-equilibrated PolyPEI column at a flow rate of 2.0 ml/min. After the injection, the column was washed with Equilibration buffer for 5 CV and the flow through product collected. Then the retained IgG aggregate was washed out with Equilibration buffer containing 1.0 M NaCl. All fractions were collected for further analysis by SEC chromatography as described in the section titled “Preparation and analysis of IgG aggregates and characterization thereof”. Based on the fraction analysis by UV, the yield of IgG monomer is 85.52% and the overall protein recovery is 99.81%. The SEC analysis of the separated monomer and aggregate solutions and the fractions thereof are shown in FIGS. 5 and 6. FIG. 4 is the SEC analysis of the injection sample of Example 2.

### Example 3

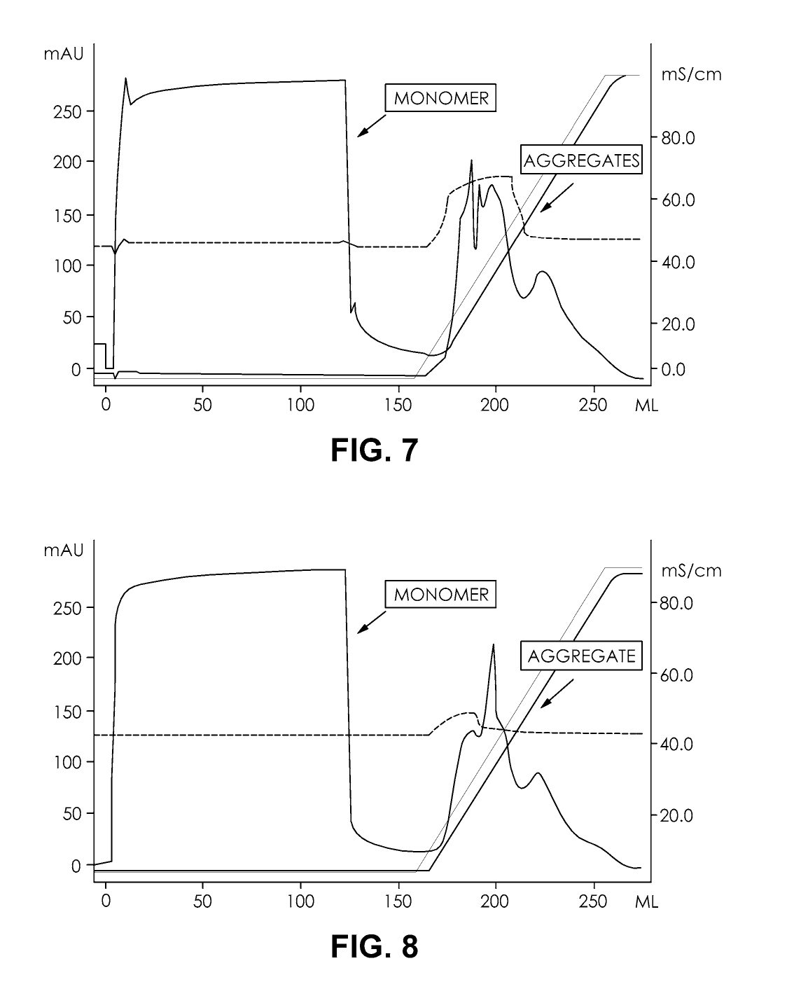

A column (100×7.75 mm ID) was packed with 35 micron BakerBond® PolyPEI. The column was equilibrated with 50 mM MES pH 6.0 buffer (Equilibration buffer) for at least 8 column volumes (CV). 120 ml of the solution containing 1.08 mg/ml IgG monomer and 0.08 mg/ml IgG aggregates in Equilibration buffer was filtered through a 0.45 μm membrane filter and then injected into the pre-equilibrated PolyPEI column at a flow rate of 2.0 ml/min. After the injection, the column was washed with Equilibration buffer for 5 CV and the flow-through product collected. Then the IgG aggregate was washed out with Equilibration buffer containing 1.0 M NaCl. All fractions were collected for further analysis by SEC chromatography as described in the section titled “Preparation and analysis of IgG aggregates and characterization thereof”. Based on the fraction analysis by UV, the yield of IgG monomer is 84.62% and the overall protein recovery is 97.33%. The SEC analysis of the separated monomer and aggregates solutions is shown in FIG. 7.

### Example 4

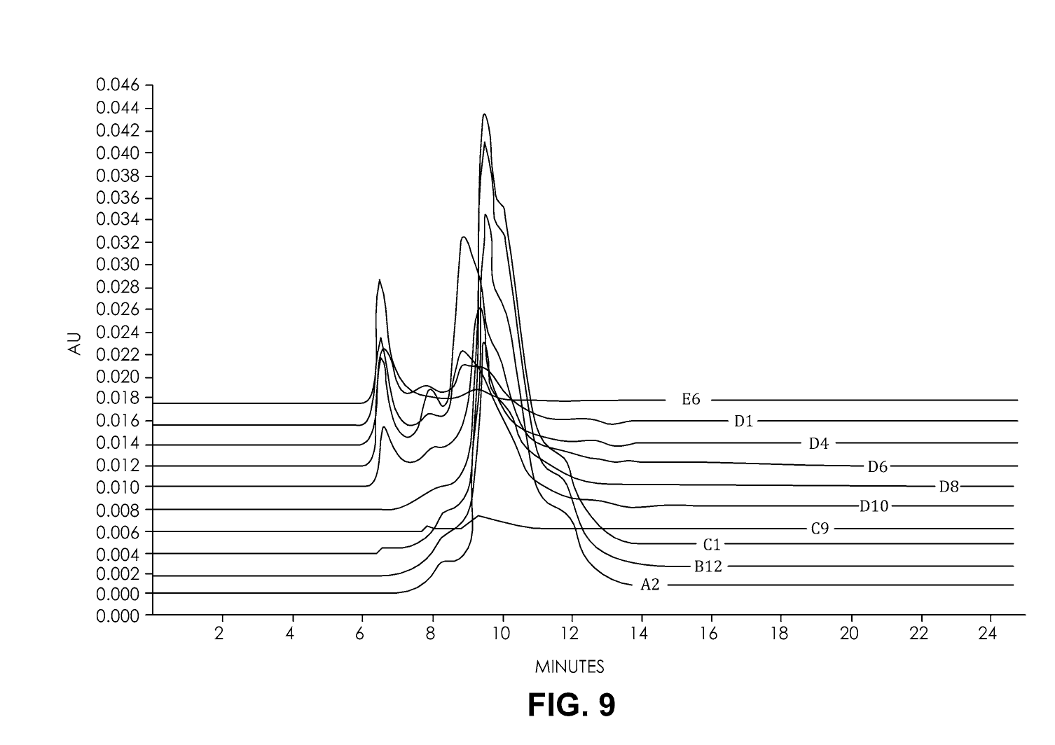

A column (100×7.75 mm ID) was packed with 35 micron BakerBond® PolyPEI. The column was equilibrated with 50 mM MES 4 mS/cm pH 6.0 buffer (Equilibration buffer) for at least 8 column volumes (CV). 120 ml of the solution containing 1.08 mg/ml IgG monomer and 0.08 mg/ml IgG aggregates in Equilibration buffer was filtered through a 0.45 μm membrane filter and then injected into the pre-equilibrated PolyPEI column at a flow rate of 2.0 ml/min. After the injection, the column was washed with Equilibration buffer for 5 CV and the flow-through product collected. Then the IgG aggregate was washed out with 50 mM MES pH 6.0 buffer containing 1.0 M NaCl. All fractions were collected for further analysis by SEC chromatography as described in the section titled “Preparation and analysis of IgG aggregates and characterization thereof”. Based on the fraction analysis by UV, the yield of IgG monomer is 89.39% and the overall protein recovery is essentially 100%. The SEC analysis is shown in FIGS. 8 and 9.

### Example 5

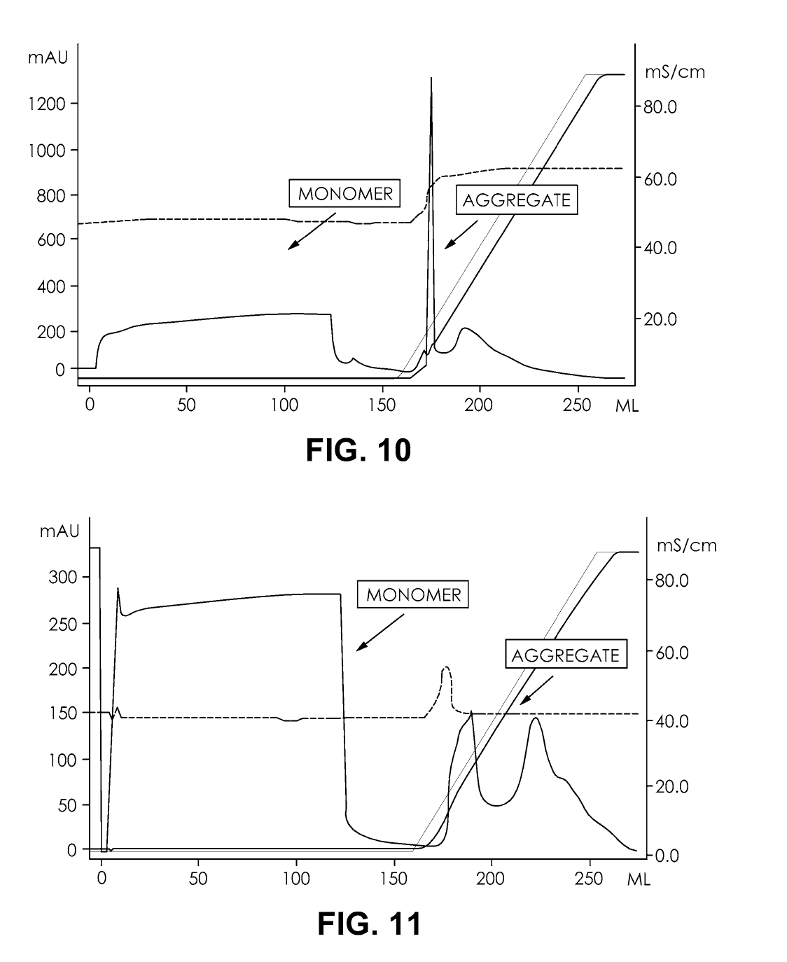

A column (100×7.75 mm ID) was packed with 35 micron BakerBond® PolyPEI. The column was equilibrated with 50 mM MES pH 7.0 buffer (Equilibration buffer) for at least 8 column volumes (CV). 120 ml of the solution containing 1.08 mg/ml IgG monomer and 0.08 mg/ml IgG aggregates in Equilibration buffer was filtered through a 0.45 μm membrane filter and then injected into the pre-equilibrated PolyPEI column at a flow rate of 2.0 ml/min. After the injection, the column was washed with Equilibration buffer for 5 CV and the flow through product collected. Then the IgG aggregates were washed out with Equilibration buffer containing 1.0 M NaCl. All fractions were collected for further analysis by SEC chromatography as described in the section titled “Preparation and analysis of IgG aggregates and characterization”. Based on the fraction analysis by UV, the yield of IgG monomer is 81.42% and the overall protein recovery is 98.69%. The SEC analysis is shown in FIG. 10.

### Example 6

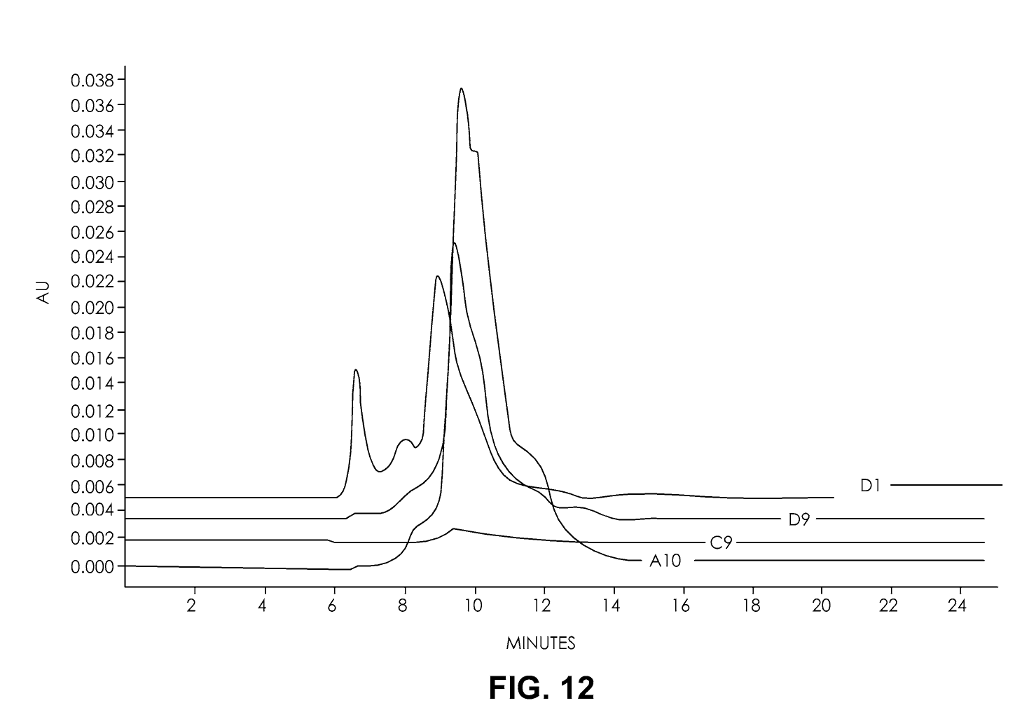

A column (100×7.75 mm ID) was packed with 40 micron XWP PEI (PN 7368). The column was equilibrated with 50 mM MES pH 6.0 buffer (Equilibration buffer) for at least 8 column volumes (CV). 120 ml of the solution containing 1.08 mg/ml IgG monomer and 0.08 mg/ml IgG aggregates in Equilibration buffer was filtered through a 0.45 μm membrane filter and then injected into the pre-equilibrated PEI column at a flow rate of 2.0 ml/min. After the injection, the column was washed with Equilibration buffer for 5 CV and the flow-through product collected. Then the IgG aggregates were washed out with Equilibration buffer containing 1.0 M NaCl. All fractions were collected for further analysis by SEC chromatography as described in the section titled “Preparation and analysis of IgG aggregates and characterization”. Based on the fraction analysis by UV, the yield of IgG monomer is 81.62% and the overall protein recovery is 92.36%. The SEC analysis is shown in FIGS. 11 and 12.

### Example 7

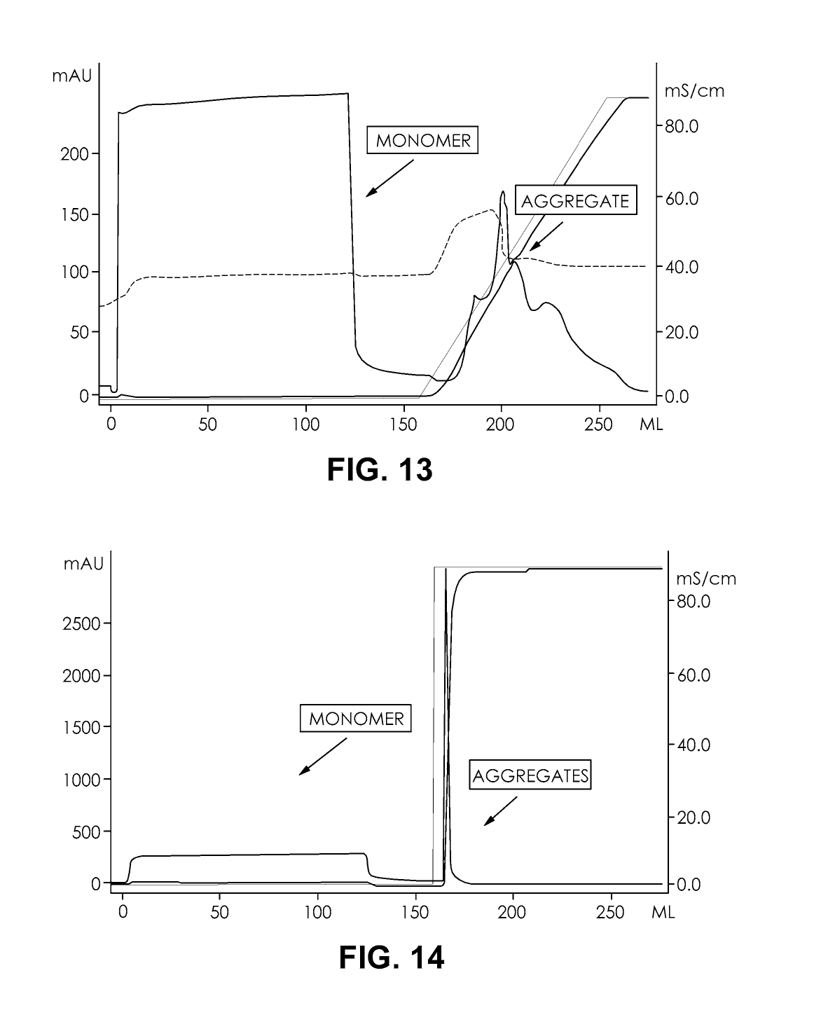

A column (100×7.75 mm ID) was packed with 35 micron BakerBond® PolyPEI. The column was equilibrated with 50 mM MES pH 5.5 buffer (Equilibration buffer) for at least 8 column volumes (CV). 120 ml of the solution containing 1.08 mg/ml IgG monomer and 0.08 mg/ml IgG aggregates in Equilibration buffer was filtered through a 0.45 μm membrane filter and then injected into the pre-equilibrated PolyPEI column at a flow rate of 2.0 ml/min. After the injection, the column was washed with Equilibration buffer for 5 CV and the flow-through product collected. Then the IgG aggregates were washed out with Equilibration buffer containing 1.0 M NaCl. All fractions were collected for further analysis by SEC chromatography as described in the section titled “Preparation and analysis of IgG aggregates and characterization”. Based on the fraction analysis by UV, the yield of IgG monomer is 89.85% and the overall protein recovery is 98.37%. The SEC analysis is shown in FIG. 13.

### Example 8

A column (100×7.75 mm ID) was packed with 35 micron BakerBond® PolyPEI. The column was equilibrated with 50 mM MES pH 6.0 buffer (Equilibration buffer) for at least 8 column volumes (CV). 120 ml of the solution containing 1.08 mg/ml IgG monomer and 0.08 mg/ml IgG aggregates in Equilibration buffer was filtered through a 0.45 μm membrane filter and then injected into the pre-equilibrated PolyPEI column at a flow rate of 2.0 ml/min. After the injection, the column was washed with Equilibration buffer for 5 CV and the flow through product collected. Then the IgG aggregate were washed out with Equilibration buffer containing 1.0 M NaCl. All fractions were collected for further analysis by SEC chromatography as described in the section titled “Preparation and analysis of IgG aggregates and characterization”. The SEC analysis is shown in FIG. 14.

If desired or considered necessary the collected purified flow-through protein monomer solution may be further processed by methods known in the art to further concentrate or convert the protein monomer to a more desirable form.

While the invention has been described herein with reference to the specific embodiments thereof, it will be appreciated that changes, modification and variations can be made without departing from the spirit and scope of the inventive concept disclosed herein. Accordingly, it is intended to embrace all such changes, modification and variations that fall with the spirit and scope of the appended claims.

We claim:

1. A flow-through process for separating protein monomer from aggregates of that protein in a solution containing both the protein monomer and the aggregates, the process comprising:
1) contacting the solution with a weak anion exchange media comprised of multiple primary, secondary and/or tertiary amine functionalization groups whereby protein monomer flows through the media without binding thereto and aggregates are retained on the media, and
2) collecting the flow-through of step 1) as purified protein monomer, and wherein the process is conducted at a pH of from pH 4 to 7.

2. The process according to claim 1 wherein the process is conducted at a pH of from pH 5.5 to 6.5.

3. The process according to claim 1 wherein the process is conducted at a pH that is less than the pI of the protein monomer being separated.

4. The process according to claim 3 wherein the process is conducted at a pH that is from 1 to 2 units below the pI of the protein monomer being separated.

5. The process according to claim 1 wherein the solution contacting the media contains a purification buffer having a conductivity of less than 10 mS/cm.

6. The process according to any claim 1 wherein the weak anion exchange media comprised of multiple primary, secondary and/or tertiary amine functionalization groups is one functionalized with a polyamine selected from the group consisting of polyethyleneimine, polyallylamine, and polyalkyleneimine.

7. The process according to claims 1 to 6 wherein the weak anion exchange media comprised of multiple primary, secondary and/or tertiary amine functionalization groups is a polymethacrylate functionalized with a polyamine.

8. The process according to claim 7 wherein the weak anion exchange media comprised of multiple primary, secondary and/or tertiary amine functionalization groups is a polymethacrylate functionalized with polyethyleneimine.

9. The process according to claim 7 wherein the weak anion exchange media comprised of multiple primary, secondary and/or tertiary amine functionalization groups is a silica functionalized with polyethyleneimine.

10. The process according to claim 1 wherein the protein monomer being separated from aggregates thereof is an immunoglobulin.

11. The process according to claim 10 wherein the immunoglobulin is IgG.

12. The process according to claim 1 wherein the weak anion exchange media functionalized with multiple primary, secondary and/or tertiary amine groups is located in a column and the solution is injected into the column.

13. The process according to claim 1 wherein an additional step of removal of the bound aggregates and other impurities with a high conductivity buffer of a conductivity of>60 mS/cm at the same pH is conducted following collecting of the purified protein monomer.
